(19) United States
(12) Patent Application Publication
(10) Pub. No.: US 2006/0222713 A1
(43) Pub. Date: Oct. 5, 2006

(54) Extended release pharmaceutical composition of phenytoin sodium

(76) Inventors: Deepak Murpani, New Delhi (IN); Ashish Madan, New Delhi (IN)

Correspondence Address: RANBAXY INC., 600 COLLEGE ROAD EAST, SUITE 2100, PRINCETON, NJ, 08540, US

(21) Appl. No.: 10/539,129

(22) PCT Filed: Dec. 16, 2003
(86) PCT No.: PCT/IB03/06007

§ 371 Date: Apr. 10, 2006

(30) Foreign Application Priority Data
Dec. 16, 2002 (IN) 1264/DEL/2002

(51) Int. Cl. A61K 9/14; A61K 31/4168

(52) U.S. Cl. 424/487; 424/488; 514/389

(57) ABSTRACT

The present invention relates to extended release pharmaceutical composition of phenytoin sodium that includes a blend of phenytoin sodium and one or more hydrophilic polymers. The blend forms a matrix after contacting an aqueous media and the matrix retains at least about 20% of the phenytoin after 1 hour. It also relates to a process for preparing the extended release pharmaceutical composition.

### FIELD OF THE INVENTION

The present invention relates to an extended release pharmaceutical composition of phenytoin sodium comprising a blend of phenytoin sodium and hydrophilic polymer(s). Also provided is a process for preparing the extended release pharmaceutical composition.

### BACKGROUND OF THE INVENTION

Phenytoin sodium is a known antiepileptic compound. Phenytoin, its sodium salt, and procedures for its manufacture are well known and disclosed in, for example, Kao et al., U.S. Pat. No. 4,696,814; Fawzi et. Al., U.S. Pat. No. 4,642,316 and Henze et al., U.S. Pat. No. 2,409,754, all of which are incorporated herein by reference.

Phenytoin sodium is commercially available as 30 mg and 100 mg capsules marketed by Parke Davis, sold under the brand name Dilantin®. These capsules contain lactose, confectioner's sugar, talc, magnesium stearate and phenytoin sodium as loose powder. The capsules are sealed with a band. Drug release problems associated with these pharmaceutical compositions have resulted in numerous recalls for failure to meet dissolution requirements. Moreover, Dilantin® requires multiple, repetitive dosing intervals. A dose of 100 mg of Dilantin requires a capsule size #3 (230 mg), therefore in order to incorporate a greater dose of the drug using Dilantin capsules to make, for example, an extended release dosage form, the size of the capsules would also have to be increased which, with respect to patient compliance, is not desirable.

Extended release oral capsules containing 200 mg and 300 mg phenytoin sodium are also available commercially under the brand name Phenylek®. These capsules contain phenytoin sodium in an erodible matrix that includes povidone, hydroxyethyl cellulose, microcrystalline cellulose, magnesium oxide, colloidal silicon dioxide and magnesium stearate as described in Mylan's U.S. Pat. No. 6,274,168 and its continuation-in-part U.S. Pat. No. 6,620,432 (prior publication: U.S. 20010043945). These extended release, oral capsules are prepared by mixing phenytoin sodium with diluents, binder(s), alkaline pH modifier(s), or a combination thereof, and then granulating with an aqueous solvent, which may or may not contain a binder(s). The dried granules are milled and finally blended with other excipients. The blend is filled into capsules or compressed into tablets. The tablets then may be additionally coated and/or filled into capsules. The pharmaceutical compositions as described by these patents are in the form of granules or tablets, which thus require the additional steps of granulation or compression, respectively.

### SUMMARY OF THE INVENTION

In one general aspect there is provided an extended release pharmaceutical composition includes a blend of phenytoin sodium and one or more hydrophilic polymers.

The blend forms a matrix after contacting an aqueous media and the matrix retains at least about 20% of the phenytoin after 1 hour.

Embodiments of the pharmaceutical composition may include one or more of the following features. For example, the composition may contain a blend of the phenytoin sodium and the hydrophilic polymers in a powder form with about 40 percent to about 70 percent by weight of the phenytoin sodium. The blend may contain about 10 percent to 30 percent by weight of the one or more hydrophilic polymers

The hydrophilic polymers may be one or more of carbohydrate gum, cellulose ether, acrylic acid polymer, and mixtures thereof. The carbohydrate gum may include one or more of xanthan gum, tragacanth gum, gum karaya, guar gum, acacia, gellan gum, locust bean gum, and mixtures thereof. Particularly, the carbohydrate gum may be xantham gum.

The cellulose ethers may be one or more of methyl cellulose, hydroxypropyl cellulose, hydroxypropyl methyl cellulose, hydroxyethyl cellulose, hydroxypropyl butyl cellulose, carboxymethyl cellulose, and combinations thereof. Particularly, the cellulose ether may be hydroxypropyl cellulose. In another embodiment the cellulose ether may be hydroxypropyl methylcellulose. The acrylic acid polymers may be carboxy vinyl polymer. The hydrophilic polymers may be a combination of a cellulose ether and carbohydrate gum. In one embodiment, the cellulose ether may be a combination of hydroxypropyl cellulose and hydroxypropyl methylcellulose and the carbohydrate gum may be xanthan gum.

The pharmaceutical composition may optionally include pharmaceutically acceptable excipients. The pharmaceutically acceptable excipients may be one or more of diluents, lubricants and glidants.

The diluents may be one or more of microcrystalline cellulose, powdered cellulose, lactose, starch, mannitol, calcium hydrogen phosphate, and dextrose. In one embodiment the diluent may be microcrystalline cellulose.

The lubricants may be one or more of talc, magnesium stearate, calcium stearate, stearic acid, hydrogenated vegetable oil, polyethylene glycol, sodium stearyl fumarate and sodium benzoate. In one embodiment the lubricant may be magnesium stearate. In another embodiment the lubricant may be talc.

The glidants may be one or both of colloidal silicon dioxide and talc. In one embodiment the glidant may be silicon dioxide.

The pharmaceutical composition may have an in vitro dissolution profile when tested using USP Apparatus I in water at 75 rpm of a) not more than about 35 percent released in about 30 minutes, b) between about 30 percent and about 75 percent released in about 60 minutes, and c) not less than about 65 percent released in about 120 minutes.

In another general aspect there is provided a process for preparing an extended release pharmaceutical composition that includes a blend of phenytoin sodium and one or more hydrophilic polymers. The process includes blending phenytoin sodium and one or more hydrophilic polymers, screening the blend, and filling the blend into capsules.

The matrix formed from the pharmaceutical composition may retain at least about 30% of phenytoin after 1 hour. In another embodiment the matrix may retain at least about 60% of phenytoin after 1 hour.

The process includes a blend of phenytoin sodium and hydrophilic polymers and may be filled into the capsule in the form of a powder. This pharmaceutical composition may include about 40 percent to about 70 percent by weight of phenytoin sodium. The pharmaceutical composition also may include from about 10 percent to about 70 percent by weight of the one or more hydrophilic polymers. The hydrophilic polymers may be one or more of carbohydrate gum, cellulose ether, acrylic acid polymer, and mixtures thereof.

The carbohydrate gum may be one or more of xanthan gum, tragacanth gum, gum karaya, guar gum, acacia, gellan gum, locust bean gum, and mixtures thereof. In one embodiment the carbohydrate gum may be xanthan gum.

The cellulose ethers may be one or more of methyl cellulose, hydroxypropyl cellulose, hydroxypropyl methyl cellulose, hydroxyethyl cellulose, hydroxypropyl butyl cellulose, carboxymethyl cellulose, and combinations thereof. In one embodiment the cellulose ether may be hydroxypropyl cellulose. In another embodiment the cellulose ether may be hydroxypropyl cellulose. The acrylic acid polymer may be carboxy vinyl polymer.

In one embodiment one or more hydrophilic polymers may include a combination of a cellulose ether and a carbohydrate gum. The cellulose ether may be a combination of hydroxypropyl cellulose and hydroxypropyl methylcellulose and the carbohydrate gum may be xanthan gum.

The mixture may be blended with one or more pharmaceutically acceptable excipients and with the phenytoin sodium and the one or more hydrophilic polymers.

The pharmaceutically acceptable excipients may be one or more of diluents, lubricants, and glidants.

The pharmaceutical composition made by this process may have the following in vitro dissolution profile when tested using USP Apparatus I in water at 75 rpm: a) not more than about 35 percent released in about 30 minutes, b) between about 30 and about 75 percent released in about 60 minutes and c) not less than about 65 percent released in about 120 minutes.

In another general aspect there is provided a method for controlling or treating one or more of generalized tonic-clonic (grand mal) seizures and complex partial (psychomotor, temporal lobe) seizures and prevention and treatment of seizures occurring during or following neurosurgery in a patient in need thereof. The method includes administering an extended-release pharmaceutical composition which includes a blend of phenytoin sodium and one or more hydrophilic polymers. The blend forms a matrix after contacting an aqueous media and the matrix retains at least about 20% of the phenytoin after 1 hour.

Embodiments of the method may contain any one or more of the features described above and one or more of the following features. For example, the pharmaceutical composition described above may also be administered with an additional pharmaceutically active agent. The additional pharmaceutically active agent may be one or both of phenobarbitone and pentobarbital.

The details of one or more embodiments of the inventions are set forth in the description below. Other features, objects and advantages of the inventions will be apparent from the description and claims.

### DETAILED DESCRIPTION

The inventors have discovered that it is possible to prepare extended release pharmaceutical compositions without performing granulation and compression, and Yet achieve batch-to-batch reproducible dissolution profiles. To accomplish this result, the inventors have formulated extended-release capsules by employing a simple process that advantageously does not involve the extra steps of granulating, drying, milling, compressing, and band-sealing after filling in capsules. This reduction in process steps likewise reduces the manufacturing costs of the dosage form. One aspect of this savings results from using a powder to fill the capsules rather than using a granulation to fill the capsules. Surprisingly, these benefits are attained while still maintaining generally reproducible extended-release properties.

Powders generally require no further treatment prior to their use and present few complicating factors when compared to granules or tablets. The main drawback of filling powders into capsules is the resulting non-reproducible in vitro release profiles. The inventors have discovered that the drug release can be regulated, and batch-to-batch variation can be minimized, by mixing or blending the active ingredient with one or more hydrophilic polymers and then filling the mixture or blend into capsules.

Phenytoin sodium is a monosodium salt of 5,5-diphenyl hydantoinate and is described on page 1259 of the Twelfth Edition of the Merck Index, which is incorporated herein by reference. It is useful as an anticonvulsant, for the treatment of generalized tonic-clonic (grand mal) seizures in adults and children, and in the treatment of simple and complex partial seizures.

Phenytoin sodium used in accordance with the present invention includes between about 40% to about 70% w/w of the total formulation weight.

Suitable pharmaceutically acceptable hydrophilic polymers used in the present pharmaceutical composition may include one or more of the carbohydrate gums, cellulose ethers, acrylic acid polymers and mixtures thereof.

Suitable carbohydrate gums maybe selected from one or more of xanthan gum, tragacanth gum, gum karaya, guar gum, acacia gellan gum, locust bean gum and the like. Upon contact with the gastrointestinal fluid, the gums form a viscous gel and sustain the release of the drug even when used in very small amounts.

Suitable cellulose ethers used in accordance with the present pharmaceutical composition include one or more of methyl cellulose, hydroxyethyl cellulose, hydroxypropyl cellulose, hydroxyethyl cellulose, hydroxypropyl methylcellulose, hydroxypropyl ethyl cellulose and hydroxypropyl butyl cellulose.

Suitable acrylic acid polymers include carboxyvinyl polymers such as those available under the brand name Carbopol (B.F. Goodrich, USA).

The one or more hydrophilic polymers may be present in an amount from about 10% to about 30% w/w of the composition. The use of small amounts of hydrophilic polymers ensures a low total weight of the dosage form and therefore provides' the therapeutic dosage of the drug in a single unit as compared to two or three units which need to be administered when using the commercially available Dilantin® 100 mg capsules. The present invention provides obvious benefits with respect to better patient convenience and therefore better patient compliance.

In addition to the active ingredient and one or more hydrophilic polymers, the composition may optionally contain one or more pharmaceutically acceptable excipients, including colorants, diluents, lubricants and glidants.

Suitable diluents may include any conventional diluents, including one or more of microcrystalline cellulose, powdered cellulose, lactose, starch, mannitol, calcium hydrogen phosphate and dextrose.

Suitable lubricants may be selected from one or more of talc, magnesium stearate, calcium stearate, polyethylene glycol, hydrogenated vegetable oils, stearic acid, sodium stearyl fumarate and sodium benzoate.

Suitable glidants may be selected from one or more of colloidal silicon dioxide (Aerosil) and talc.

The process for manufacturing the pharmaceutical composition includes blending the active ingredient, polymer and optional excipient(s) using one or more of tumbler mixers, ribbon mixers, twin shell V-blenders, double cone blenders, planetary mixers, and fluid bed mixers. The resulting blend is then filled into hard gelatin capsules using either gravity, wherein the powder blend is filled into the capsule due to its natural flow, or partial compression, wherein weak slugs are formed inside a calibrated punch prior to being deposited into the capsule.

Optionally, the lubricants and glidants may be added after thorough blending of other components of the formulation. This blend is passed through a No. 30 mesh screen and filled into capsules, e.g., hard gelatin capsules.

The extended release phenytoin sodium capsules maintain a stable dissolution profile after storage for 3 months at 40° C. and 75% relative humidity over a two hour period when measured in vitro by dissolution testing. Dissolution testing is carried out in 900 ml of water using USP Dissolution Apparatus I (basket) at 50 rpm (for 100 mg capsules) and 75 rpm (for 200/300 capsules). The 100 mg capsules formulated as described herein show the following in vitro active ingredient dissolution profile: (a) not more than about 35 percent released in about 30 minutes, (b) not more than about 75 percent released in about 60 minutes, and (c) not less than about 65 percent released in about 120 minutes. The 200 mg and 300 mg capsules show the following in vitro dissolution profile: (a) not more than about 40 percent released in about 30 minutes, (b) not more than about 65 percent released in about 60 minutes, and (c) not less than about 75 percent released in about 120 minutes.

The extended release capsules of phenytoin sodium described herein provide a method for the control of generalized tonic-clonic (grand mal) seizures and complex partial (psychomotor, temporal lobe) seizures. Additional indications include administering the capsules for the prevention and/or treatment of seizures occurring during or following neurosurgery to a patient in need thereof. Such a method includes administering extended-release pharmaceutical compositions that include a blend of phenytoin sodium and one or more hydrophilic polymers.

The extended-release pharmaceutical composition may also include one or more additional active ingredients combined into a single pharmaceutical composition. Suitable additional active ingredients may include phenobarbitone and pentobarbital.

The following examples further exemplify the invention and are not intended to limit the scope of the invention.

### EXAMPLES 1-5

| | mg/Capsule | | | | |
|---|---|---|---|---|---|
| Ingredients | Ex 1 | Ex 2 | Ex 3 | Ex 4 | Ex 5 |
| Phenytoin | 300.0 | 300.0 | 300.0 | 300.0 | 300.0 |
| sodium | | | | | |
| Xanthan gum | 20.0 | 25.0 | 20.0 | 20.0 | 20.0 |
| Hydroxypropyl | 25.0 | 35.0 | 20.0 | — | 30.0 |
| cellulose | | | | | |
| Hydroxypropyl | 75.0 | 90.0 | 80.0 | 100.0 | 55.0 |
| methylcellulose | | | | | |
| Microcrystalline | 18.75 | 18.75 | 18.75 | 18.75 | 18.75 |
| cellulose | | | | | |
| Talc | 15.0 | 15.0 | 15.0 | 15.0 | 15.0 |
| Colloidal silicon | 1.25 | 1.25 | 1.25 | 1.25 | 1.25 |
| dioxide | | | | | |
| Magnesium stearate | 10.0 | 10.0 | 10.0 | 10.0 | 10.0 |

Process:

Phenytoin sodium, microcrystalline cellulose, hydroxypropyl cellulose, xanthan gum and hydroxypropyl methylcellulose are loaded into a twin shell V-blender and blended. Talc, colloidal silicon dioxide and magnesium stearate then are added to the blend, and mixed. This blend is screened through a No. 30 mesh screen and filled into size “0” hard gelatin capsules using automatic capsule filling machines. These capsules were then packed into high-density polyethylene bottles and stored for 3 months at 40° C. and 75% relative humidity and tested for in-vitro dissolution. Table 1 shows the dissolution data of Phenytoin sodium 300 mg capsules prepared as per the composition of Example 3 at the initial time before storage and after storage for 3 months at 40° C. and 75% relative humidity.

TABLE 1
In vitro dissolution profile of Phenytoin sodium capsules
using USP Apparatus I/900 ml water/75 rpm

Percent phenytoin sodium released (%)

| | | After storage for 3 months |
|---|---|---|
| Time (min) | Initial | at 40° C./75% RH |
| 30 | 20.0 | 22.0 |
| 60 | 42.0 | 44.0 |
| 120 | 73.0 | 79.0 |

As illustrated in Table 1, the dosage form releases the active ingredient as follows: about 20-30% after 30 minutes, about 40-50% after 60 minutes, and about 75-85% after 120 minutes.

### EXAMPLE 6

| Ingredients | mg/Capsule |
|---|---|
| Phenytoin sodium | 100.0 |
| Xanthan gum | 6.7 |
| Hydroxypropyl cellulose | 6.7 |
| Hydroxypropyl methylcellulose | 26.7 |
| Microcrystalline cellulose | 6.25 |
| Talc | 5.0 |
| Colloidal silicon dioxide | 0.42 |
| Magnesium stearate | 3.33 |

Process:

Phenytoin sodium, microcrystalline cellulose, hydroxypropyl cellulose, xanthan gum and hydroxypropyl methylcellulose are loaded into a twin shell V-blender and blended. Talc, colloidal silicon dioxide and magnesium stearate are added to the blend and mixed. This blend is screened through a No. 30 mesh screen and filled into size “0” hard gelatin capsules using automatic capsule filling machines. These capsules then are packed into high-density polyethylene bottles and stored for 3 months at 40° C. and 75% relative humidity and tested for in-vitro dissolution.

Table 2 shows the dissolution data of Phenytoin sodium 100 mg capsules prepared as per the composition of Example 6 using USP Apparatus 1,900 ml water at 50 and 75 RPM.

TABLE 2
In vitro dissolution profile of Phenytoin sodium
capsules using USP Apparatus I/900 ml water.

Phenytoin sodium released (%)

| Time (min) | At 50 RPM | At 75 RPM |
|---|---|---|
| 30 | 33.0 | 45.0 |
| 60 | 71.0 | 79.0 |
| 90 | 91.0 | 93.0 |
| 120 | 97.0 | 95.0 |

As illustrated in Table 2, at 50 RPM the dosage form releases the active ingredient as follows: about 30-40% after 30 minutes, about 65-75% after 60 minutes, about 87-94% after 90 minutes, and about 93-99% after 120 minutes.

While several particular forms of the invention have been illustrated and described, it will be apparent that various modifications and combinations of the invention detailed in the text can be made without departing from the spirit and scope of the invention. For example, an extended release capsule can be formulated that consists essentially of a simple blend or mixture of phenytoin sodium and one or more hydrophilic polymers, such as those described above. Other nonessential ingredients optionally can be added to the blend or mixture for cosmetic, aesthetic and/or manufacturing purposes. These include colorants, diluents, lubricants, and glidants. Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. An extended release pharmaceutical composition in the form of a capsule comprising:
a powder blend of phenytoin sodium; and
one or more hydrophilic polymers; wherein the hydrophilic polymers comprise a combination of a cellulose ether and carbohydrate gum.

2. The composition according to claim 49, wherein the matrix retains at least about 30% of the phenytoin after 1 hour.

3. The composition according to claim 49, wherein the matrix retains at least about 60% of the phenytoin after 1 hour.

4. (canceled)

5. (canceled)

6. The composition according to claim 1, wherein the composition comprises from about 40 percent to about 70 percent by weight of phenytoin sodium.

7. The composition according to claim 1, wherein the composition comprises from about 10 percent to about 30 percent by weight of the one or more hydrophilic polymers.

8. (canceled)

9. The composition according to claim 1, wherein the carbohydrate gum comprises one or more of xanthan gum, tragacanth gum, gum karaya, guar gum, acacia, gellan gum, locust bean gum, and mixtures thereof.

10. The composition according to claim 9, wherein the carbohydrate gum comprises xanthan gum.

11. The composition according to claim 1, wherein the cellulose ether comprises one or more of methyl cellulose, hydroxypropyl cellulose, hydroxypropyl methyl cellulose, hydroxyethyl cellulose, hydroxypropyl butyl cellulose, carboxymethyl cellulose, and combinations thereof.

12. The composition according to claim 11, wherein the cellulose ether comprises hydroxypropyl cellulose.

13. The composition according to claim 11, wherein the cellulose ether comprises hydroxypropyl methylcellulose.

14. (canceled)

15. (canceled)

16. The composition according to claim 1, wherein the cellulose ether comprises a combination of hydroxypropyl cellulose and hydroxypropyl methylcellulose and the carbohydrate gum comprises xanthan gum.

17. The composition according to claim 1, further comprising one or more pharmaceutically acceptable excipients.

18. The composition according to claim 17, wherein the one or more pharmaceutically acceptable excipients comprise one or more of diluents, lubricants and glidants.

19. The composition according to claim 18, wherein the diluents comprise one or more of microcrystalline cellulose, powdered cellulose, lactose, starch, mannitol, calcium hydrogen phosphate, and dextrose.

20. The composition according to claim 19, wherein the diluent comprises microcrystalline cellulose.

21. The composition according to claim 18, wherein the lubricant comprises one or more of talc, magnesium stearate, calcium stearate, stearic acid, hydrogenated vegetable oil, polyethylene glycol, sodium stearyl fumarate and sodium benzoate.

22. The composition according to claim 21, wherein the lubricant comprises magnesium stearate.

23. The composition according to claim 21, wherein the lubricant comprises talc.

24. The composition according to claim 18, wherein the glidant comprises one or more of colloidal silicon dioxide and talc.

25. The composition according to claim 24, wherein the glidant comprises colloidal silicon dioxide.

26. The composition according to claim 1, wherein the composition has the following in vitro dissolution profile when tested using USP Apparatus I in water at 75 rpm:
a) not more than about 35 percent released in about 30 minutes,
b) between about 30 percent and about 75 percent released in about 60 minutes, and
c) not less than about 65 percent released in about 120 minutes.

27. A process for preparing an extended release pharmaceutical composition comprising a blend of phenytoin sodium and one or more hydrophilic polymers; the process comprising;
a) blending phenytoin sodium and one or more hydrophilic polymers,
b) screening the blend, and
c) filling the blend into capsules.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. A method for controlling or treating one or more of generalized tonic-clonic (grand mal) seizures and complex partial (psychomotor, temporal lobe) seizures and prevention and treatment of seizures occurring during or following neurosurgery in a patient in need thereof, the method comprising administering an extended-release pharmaceutical composition comprising:
a powder blend of phenytoin sodium; and
hydrophilic polymers;
wherein the hydrophilic polymers comprise a combination of a cellulose ether and carbohydrate gum.

46. The method according to claim 45, further comprising administering an additional pharmaceutically active agent.

47. The method according to claim 46, wherein the additional pharmaceutically active agent comprises one or both of phenobarbitone and pentobarbital.

48. The method according to claim 45, wherein the one or more hydrophilic polymers comprise one or more of carbohydrate gum, cellulose ether, acrylic acid polymer, and mixtures thereof.

49. The composition according to claim 1, wherein the powder blend forms a matrix after contacting an aqueous media and the matrix retains at least about 20% of the phenytoin after 1 hour.
